(19) United States
(12) Patent Application Publication
(10) Pub. No.: US 2026/0306771 A1
(43) Pub. Date: Oct. 8, 2026

(54) VENTILATION ARRANGEMENT WITH AN ANESTHETIC DOSING UNIT AND A PNEUMATIC SWITCH

(71) Applicant: Drägerwerk AG & Co. KGaA, Lübeck (DE)

(72) Inventors: Ralf HEESCH, Lübeck (DE); Jana TOMAT, Lübeck (DE)

(21) Appl. No.: 19/634,387

(22) Filed: Mar. 31, 2026

(30) Foreign Application Priority Data
Apr. 2, 2025 (DE) 10 2025 112 825.1

(51) Int. Cl. A61M 16/00; A61M 16/18

(52) U.S. Cl. CPC A61M 16/022; A61M 16/0003; A61M 16/18; A61M 2205/3368

(57) ABSTRACT

A ventilation arrangement and a ventilation process are configured to artificially ventilate a patient. The patient is supplied with a ventilation gas mixture (Bg) including an anesthetic gas mixture (Ng). An anesthetic dosing unit (20) provides the anesthetic gas mixture (Ng). A dosing specification is captured which specifies a desired anesthetic concentration in the anesthetic gas mixture (Ng). After capturing the dosing specification, the anesthetic gas mixture (Ng) is first discharged into a sink (10). The anesthetic gas mixture (Ng) is only fed into an inspiratory fluid guide unit (3.1) and conveyed as part of the ventilation gas mixture (Bg) to a patient-side coupling unit if the actual concentration matches the desired anesthetic concentration. A controlled pneumatic switch (30) directs the anesthetic gas mixture (Ng) either to the inspiratory fluid guide unit (3.1) or to the sink (10).

### CROSS REFERENCE TO RELATED APPLICATIONS

This application claims the benefit of priority under 35 U.S.C. § 119 of German Application 10 2025 112 825.1, filed Apr. 2, 2025, the entire contents of which are incorporated herein by reference.

### TECHNICAL FIELD

The invention relates to a ventilation arrangement comprising an anesthetic dosing unit (anesthetic dispenser), a pneumatic switch (pneumatic diverter valve), and an inspiratory fluid guide unit leading to a patient-side coupling unit, the ventilation arrangement being configured to artificially ventilate (ventilate) and anesthetize a patient. The invention also relates to a ventilation system. Furthermore, the invention relates to a ventilation process that is carried out using such a ventilation arrangement.

### SUMMARY

It is an object of the invention to provide a ventilation arrangement comprising an anesthetic dosing unit and an inspiratory fluid guide unit to a patient-side coupling unit and, in some situations, configured for supplying, with a higher degree of reliability than known ventilation arrangements, a patient with an anesthetic gas mixture which is to have a desired property. It is another object of the invention to provide a ventilation system for supplying a patient with a higher degree of reliability than known ventilation systems with an anesthetic gas mixture which is to have a desired property. It is furthermore an object of the invention to provide a ventilation process that is carried out using such a ventilation arrangement and that has a higher reliability than known ventilation processes.

These objects are attained by a ventilation arrangement and by a ventilation system with features according to this disclosure and by a ventilation process with features according to this disclosure. Advantageous embodiments are specified in this disclosure including the text, drawings and the claims. Advantageous embodiments of the ventilation arrangement according to the present disclosure are, where appropriate, also advantageous embodiments of the ventilation process according to the present disclosure and vice versa.

The ventilation arrangement according to the present disclosure is configured to artificially ventilate a patient and to supply him/her with a ventilation gas mixture. The ventilation gas mixture comprises oxygen and at least one anesthetic. The ventilation process according to the present disclosure is used to artificially ventilate a patient with such a ventilation gas mixture. Because the ventilation gas mixture contains at least one anesthetic, the patient is anesthetized at least temporarily during artificial ventilation.

The ventilation arrangement according to the present disclosure comprises a patient-side coupling unit. The patient-side coupling unit is configured to be positioned in and/or on the body of the artificially ventilated patient. A breathing mask and a tube are two examples of a patient-side coupling unit. The ventilation arrangement supplies the ventilation gas mixture to the patient-side coupling unit so that the patient can inhale it. While the ventilation process according to the present disclosure is being performed, the patient-side coupling unit is positioned in or on the patient's body.

The ventilation arrangement further comprises
an anesthetic dosing unit with a dosing unit outlet,
a carrier gas fluid guide unit,
a supply fluid guide unit,
a pneumatic switch,
an inspiratory fluid guide unit, and
a discharge fluid guide unit.

The pneumatic switch comprises a switch inlet, a supply outlet, and a discharge outlet. The switch can be controlled and, depending on the control signal, switches to a supply position and to a discharge position or is moved to these positions. In the supply position, the switch establishes a fluid connection between the switch inlet and the supply outlet and preferably separates the discharge outlet from the switch inlet in a fluid-tight manner. In the discharge position, the switch establishes a fluid connection between the switch inlet and the discharge outlet and preferably separates the supply outlet from the switch inlet in a fluid-tight manner.

The anesthetic dosing unit is configured for generating an anesthetic gas mixture. The anesthetic gas mixture comprises a carrier gas (fresh gas) and at least one anesthetic. The carrier gas preferably comprises at least one of the three components breathing air, pure oxygen, and nitrous oxide (N₂O). Optionally, the anesthetic gas mixture additionally comprises at least one gaseous medication. The anesthetic dosing unit provides the anesthetic gas mixture at its dosing unit outlet.

The carrier gas fluid guide unit connects a carrier gas source to the anesthetic dosing unit. In one embodiment, the carrier gas source comprises a carrier gas mixer that composes the carrier gas from at least two components. The carrier gas source may additionally comprise at least one stationary supply connection, wherein the one supply connection or each supply connection preferably provides the carrier gas or a carrier gas component under overpressure.

A first segment of the supply fluid guide unit connects the dosing unit outlet to the inlet of the switch. A second segment of the supply fluid guide unit connects the supply outlet of the switch to a feed-in point (injection point). The discharge fluid guide unit connects the discharge outlet of the switch to a sink, in particular to a waste gas processor, in particular one for anesthetics.

The inspiratory fluid guide unit connects the feed-in point to the patient-side coupling unit.

Note: A “fluid guide unit” is understood to be a component that is configured for conducting (guiding) a fluid, in particular a gas mixture, along a trajectory. The trajectory is predetermined by the design and arrangement of the fluid guide unit. Ideally, the fluid guide unit completely prevents the guided fluid from leaving the trajectory laterally. A tube and a hose are two examples of a fluid guide unit. A fluid conveying unit is configured for delivering (conveying) a fluid, in particular into a fluid guide unit or along a fluid guide unit. A pump, a blower, and a piston-cylinder unit are examples of a fluid conveying unit.

The ventilation arrangement is configured to perform the following steps, and the ventilation process comprises the following steps:
The carrier gas is conveyed or otherwise directed (transported) from the carrier gas source through the carrier gas fluid guide unit to the anesthetic dosing unit.
The anesthetic dosing unit generates the anesthetic gas mixture and makes it available at its dosing unit outlet.
The anesthetic gas mixture is conveyed or otherwise directed through the first segment of the supply fluid guide unit to the inlet of the switch.

While the pneumatic switch is in the supply position, the following additional steps are performed:
The anesthetic gas mixture is conveyed or otherwise directed through the switch to the supply outlet and through the second segment of the supply fluid guide unit to the feed-in point, and is fed into the inspiratory fluid guide unit at the feed-in point.
The ventilation gas mixture with the fed-in anesthetic gas mixture is conveyed or otherwise directed through the inspiratory fluid guide unit to the patient-side coupling unit.

While the switch is in the discharge position, the following additional step is performed: The anesthetic gas mixture is conveyed or otherwise directed through the pneumatic switch to the discharge outlet and from there through the discharge fluid guide unit to the sink.

The ventilation arrangement is configured for capturing (acquiring) a dosing specification (dosage setting), and the ventilation process comprises the step of capturing a dosing specification at least once. The or each captured dosing specification specifies a respective desired concentration of the anesthetic in the anesthetic gas mixture to be generated and fed in. The anesthetic dosing unit is configured for generating the anesthetic gas mixture with the objective (control goal) of ensuring that the actual anesthetic concentration in the anesthetic gas mixture is equal to (coincides with) the desired anesthetic concentration. More precisely, the actual anesthetic concentration should not deviate from the desired anesthetic concentration by more than a given absolute or percentage tolerance. It is possible that the desired anesthetic concentration in the anesthetic gas mixture results from a given desired anesthetic concentration in the breathing gas mixture reaching the patient-side coupling unit.

The ventilation arrangement is configured to perform the following steps in response to capturing the dosing specification, and the step that the dosing specification has been captured triggers the following steps:
The switch is switched to the discharge position or held in the discharge position. Preferably, a signal-processing control unit causes the switch to be switched to the discharge position by means of a corresponding control signal.
It is measured or otherwise determined whether a target situation exists (actually occurs). Preferably, a check is performed repeatedly to determine whether the target situation exists. While this check is being performed, the switch is in the discharge position.
The target situation is present if the following situation has occurred: The actual anesthetic concentration at a reference point deviates from the desired anesthetic concentration by no more than the given tolerance. The reference point is arranged in the first segment of the supply fluid guide unit, is arranged downstream of the dosing unit outlet and is preferably the inlet of the pneumatic switch, but may also be arranged upstream of this inlet. The desired anesthetic concentration is specified in the captured dosing specification.

The ventilation arrangement is configured to perform the following steps after the detection that the target situation exists (occurs, has been reached), and the ventilation process comprises the following steps, which are performed after this determination:
The switch is switched to the supply position. Preferably, the control unit causes the switch to be switched to the supply position by means of a corresponding control signal.
This step directs the anesthetic gas mixture from the inlet to the supply outlet.

The invention takes into account the following facts:
After the dosing specification has been captured, the anesthetic dosing unit is in general not able to immediately provide the anesthetic gas mixture with the desired anesthetic concentration. Even with a properly functioning anesthetic dosing unit, there is a response time that elapses before the actual anesthetic concentration at the dosing unit outlet deviates from the desired anesthetic concentration by no more than the tolerance.
The first segment, and thus also the section in the first segment from the dosing unit outlet to the reference point, inevitably has a certain pneumatic dead time. Only after this dead time has elapsed, there is no longer any gas mixture in the first segment that was already present there before the dosing specification was captured, i.e., a mixture that often has the wrong anesthetic concentration.
Between the event that the anesthetic gas mixture with the desired anesthetic concentration is delivered to the dosing unit outlet and the event that the anesthetic gas mixture reaches the desired concentration at the reference point, a certain time span inevitably elapses (a time lag). This time span is called the flow time.

Thanks to the present disclosure, the anesthetic gas mixture is only fed to the feed-in point and fed into the inspiratory fluid guide unit if the target situation has positively been detected or determined in some other way. This generally ensures that the anesthetic gas mixture also has the desired anesthetic concentration at the feed-in point. As long as the target situation has not yet been determined, the anesthetic gas mixture is not directed to the feed-in point, but to the sink. In other words, the anesthetic gas mixture is only directed to the feed-in point once it has been positively determined that it has the desired anesthetic concentration. In other words, the switch is initially in the discharge position and is only switched to the supply position if it has been positively determined that the target situation exists. This leads to greater operational safety compared to a procedure in which the switch is initially in the supply position and is only switched to the discharge position if an impermissible anesthetic concentration has been detected. With this procedure, an anesthetic concentration outside the target range is often only discovered later. Thanks to the invention, often a fail-safe situation is achieved if the pneumatic switch or a drive for the pneumatic switch fails, e.g. the switch stuck in a position. It is accepted that in some cases the consumption of anesthetic is greater with the process according to the invention.

The effect of the invention is particularly advantageous for the following reason: Closed-loop control is often used for the anesthetic dosing unit. The control objective in this control system is to ensure that the actual anesthetic concentration in the anesthetic gas mixture supplied by the anesthetic dosing unit at the dosing unit outlet is equal to the desired anesthetic concentration within a certain given tolerance. An additional or alternative control objective may be that the anesthetic concentration in the ventilation gas mixture is equal to a specified anesthetic concentration. For this control, the current actual anesthetic concentration is usually measured at a measuring position downstream of the dosing unit outlet, in particular at a measuring position in the supply fluid guide unit or in the inspiratory fluid guide unit. The anesthetic dosing unit is controlled with the objective of reducing a control deviation. However, as just explained, a certain amount of time inevitably elapses until the desired anesthetic concentration is actually reached at the measuring position. In ventilation arrangements known from the prior art, the control system sometimes leads to the undesirable event that the actual anesthetic concentration oscillates or even overshoots after the dosing specification has been captured. These oscillations result in particular from the fact that the control system sometimes overcompensates for the control deviation. The invention reduces the risk of these undesirable oscillations, in particular by directing an anesthetic gas mixture with a significantly different anesthetic concentration to the sink rather than to the feed-in point.

Note: The wording used is that a sensor is configured for measuring a physical variable (or that a sensor is configured to measure a physical variable), for example, the concentration of an anesthetic in a gas mixture. This wording means that the sensor is configured for directly measuring the physical variable or at least another quantity that correlates with the quantity to be measured. The other variable is, for example, the intensity of electromagnetic radiation that is attenuated by the target gas and hits a measuring detector, or an electrical charge that flows as a result of a chemical reaction. The measured variable or other quantities, or the combination of the measured quantities, are therefore an indicator for the physical quantity to be measured. The measurement provides at least one value for the physical variable sought.

In many cases, the invention leads to the following desired effect: The switch is only in the discharge position for as long as necessary and in the supply position for as long as possible. As a result, no anesthetic gas mixture or an anesthetic gas mixture with a significantly different anesthetic concentration is fed into the feed-in point for only a short time span.

According to the present disclosure, the step of capturing the dosing specification triggers the steps of
switching the pneumatic switch to the discharge position and
determining whether the target situation exists.

In one embodiment, these steps are always performed after the dosing specification has been captured. In another embodiment, these steps are only performed if the following situation exists: The desired anesthetic concentration specified in the captured dosing specification deviates by more than a given absolute or percentage threshold from the anesthetic concentration that the anesthetic gas mixture provided and fed into the system had before the dosing specification was captured. In other words, these two steps are only performed if the dosing specification specifies a significant change in the anesthetic concentration.

According to the present disclosure, it is measured or otherwise determined whether or not the target situation exists. As already mentioned, the target situation exists (occurs) if the actual anesthetic concentration at the reference point deviates from the desired anesthetic concentration by no more than the given tolerance. Three different embodiments for determining whether the target situation exists are described below.

According to the first embodiment, the ventilation arrangement comprises an anesthetic sensor. The anesthetic sensor is configured for measuring the concentration of the anesthetic in the anesthetic gas mixture. The measured anesthetic concentration refers to a measuring position. The measuring position is arranged in the first segment of the supply fluid guide unit, i.e., between the dosing unit outlet and the inlet of the switch. The measuring position can act as the reference point. It is possible that the anesthetic sensor extracts a gas sample from the first segment, examines it, and preferably feeds it back into the first segment. It is also possible that a measuring chamber of the anesthetic sensor is arranged in the first segment, meaning that a section of the first segment itself acts as the measuring chamber. This embodiment eliminates the need to extract and refeed a gas sample for analysis. In particular, this implementation saves time and sometimes also installation space. The risk of a leak occurring is reduced.

The ventilation arrangement is configured as follows, and the ventilation process comprises the following step: Whether or not the target situation exists is determined depending on the measured actual anesthetic concentration at the measuring position. In other words, an automatic decision is made as to whether or not the target situation currently exists. It is preferred that a determination is made that the target situation actually exists if the actual concentration of anesthetic at the measuring position does not deviate from the desired concentration by more than the tolerance.

The embodiment with the anesthetic sensor in or on the first segment eliminates the need to measure or otherwise determine the volume flow through the carrier gas fluid guide unit or the volume flow through the supply fluid guide unit. This embodiment also depends only slightly, ideally not at all, on the pneumatic properties of a fluid guide unit used for guiding gas.

The alternative embodiment described below eliminates the need to use an anesthetic sensor in or on the first segment. Instead, a delay time span (a delay time period or time lag) is determined or predicted. The target situation exists after the delay time span has elapsed since the dosing specification was captured. The following describes how the delay time span is determined or predicted.

According to the alternative embodiment, the volume flow through the carrier gas fluid guide unit or the volume flow through the supply fluid guide unit is measured or determined in another way. At least one delay parameter (delay variable) is determined. The or each delay parameter causes the anesthetic gas mixture at the reference point to exhibit the desired anesthetic concentration only after a delay. At least one of the following three delay parameters is determined:
the response time of the anesthetic dosing unit at the determined volume flow,
the pneumatic dead time at the determined volume flow in a section in the first segment, the section leading from the dosing unit outlet to the reference point,
the flow time of the anesthetic gas mixture through this section at the determined volume flow.

The delay time span is determined depending on the or each determined delay parameter. In one embodiment, the pneumatic dead time is determined as the only delay parameter, and preferably the delay time span used for the prediction is equal to the determined pneumatic dead time. This is because the pneumatic dead time usually causes a greater delay than the other two delay parameters. In another embodiment, at least two delay parameters are determined, preferably the pneumatic dead time and another delay parameter. The delay time span is preferably the sum of the determined delay parameters.

In a third embodiment, the ventilation arrangement comprises the anesthetic sensor as described above with reference to the first embodiment. The reference point is arranged downstream of the measuring position and is preferably the same as the inlet of the switch. Therefore, a pneumatic dead time and a flow time occur in the section between the measuring position and the reference point. The ventilation arrangement is configured for measuring or otherwise determining the volume flow of the carrier gas. The ventilation arrangement is configured for determining the pneumatic dead time and/or the flow time at the determined volume flow, i.e., two of the three delay parameters just mentioned. Because the actual anesthetic concentration is measured at the measuring position, it is not necessary to determine the response time of the anesthetic dosing unit. The target situation exists if the following situation has occurred and is detected:
The actual anesthetic concentration at the measuring position deviates from the desired anesthetic concentration by no more than the tolerance.
Subsequently, i.e., since the event that this broad agreement was detected, a delay time span has elapsed. The delay time span depends on the determined pneumatic dead time and/or the determined flow time.

The third embodiment further increases the reliability that only an anesthetic gas mixture with the desired anesthetic concentration is actually fed at the feed-in point.

In one embodiment, the ventilation arrangement is configured to additionally perform the following step in response to capturing the dosing specification, and the step that the dosing specification has been captured triggers the following additional step in this embodiment: A volume flow with which the carrier gas flows through the carrier gas fluid guide unit to the anesthetic dosing unit is increased. Alternatively, a volume flow with which the anesthetic gas mixture flows from the dosing unit outlet through the first segment of the supply fluid guide unit to the switch is increased. Preferably, the volume flow is increased to a given maximum achievable value.

In a further development of this embodiment, the ventilation arrangement and the ventilation process are configured as follows: After determining that the target situation exists, additionally the following step is performed: The volume flow of the carrier gas and/or the volume flow of the anesthetic gas mixture is reduced again. It is particularly preferred that the volume flow is reduced to the value prior to capturing of the dosing specification.

According to the embodiment just described, the volume flow of the carrier gas through the carrier gas fluid guide unit and/or the volume flow of the anesthetic gas mixture through the supply fluid guide unit is increased after the dosing specification has been captured. In particular, this step achieves the following effects compared to an embodiment in which the volume flow remains unchanged:
The first segment of the supply fluid guide unit is flushed out more quickly. Any anesthetic gas mixture or other gas mixture that was already present in the first segment when the dosing specification was captured is removed from the first segment more quickly. The pneumatic dead time is thus reduced.
As a rule, the above-mentioned response time of the anesthetic dosing unit is the shorter, the greater the volume flow of the carrier gas through the anesthetic dosing unit is, all other conditions being equal. An increased volume flow therefore results in a shorter response time. The dosing specification is implemented more quickly.
In addition, the flow time is generally shorter with a higher volume flow.

As a result, an anesthetic gas mixture with a desired anesthetic concentration usually reaches the patient-side coupling unit faster than if the volume flow had remained unchanged.

The ventilation arrangement preferably implements a ventilation circuit. An expiratory fluid guide unit returns the air that the patient has exhaled into the patient-side coupling unit back to the inspiratory fluid guide unit. The exhaled air usually contains anesthetic. The ventilation circuit prevents, ideally completely, anesthetic from escaping into the environment.

The invention is described below with reference to exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a ventilation circuit;

FIG. 2 is a sectional detailed view of the ventilation circuit from FIG. 1 with an anesthetic dosing unit and a pneumatic switch;

FIG. 3 is a schematic view of an embodiment of the anesthetic sensor;

FIG. 4 is a diagram showing how the step response of the anesthetic dosing unit depends on the volume flow of the carrier gas;

FIG. 5 is a flowchart illustrating a first embodiment of the invention;

FIG. 6 is a flowchart illustrating a second embodiment of the invention; and

FIG. 7 is a flowchart illustrating a third embodiment of the invention.

### DESCRIPTION OF PREFERRED EMBODIMENTS

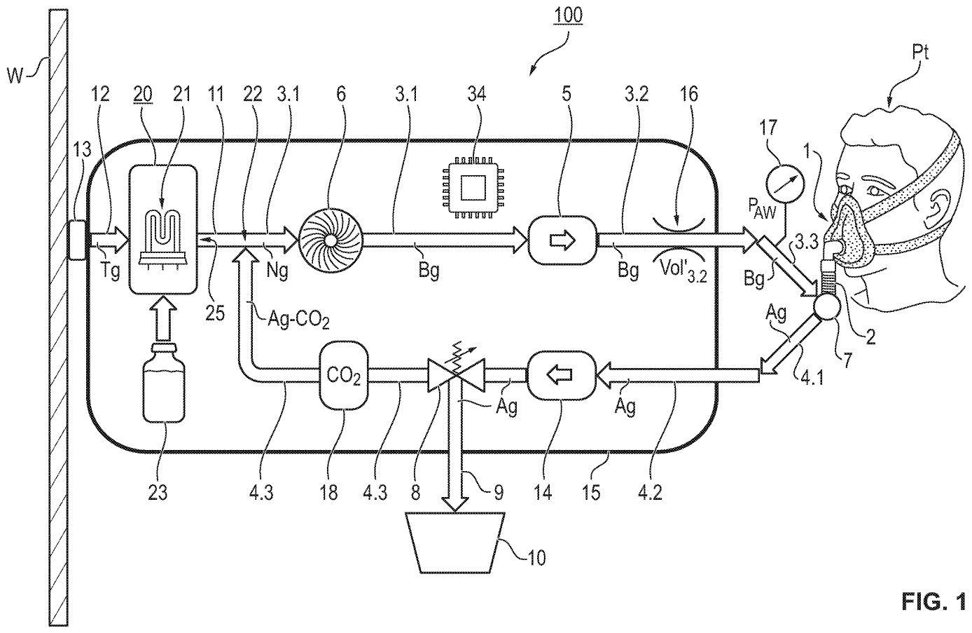

Referring to the drawings, FIG. 1 schematically shows a ventilation arrangement. A patient Pt is artificially ventilated and is anesthetized during artificial ventilation (also known as ventilation). A patient-side coupling unit 1 is arranged on and/or in the body of the patient Pt, in the shown example a breathing mask. A ventilation gas mixture Bg is conveyed to the patient-side coupling unit 1 and can be inhaled by the patient Pt. The ventilation gas mixture Bg comprises oxygen and at least one anesthetic, optionally additionally at least one gaseous medication. The air Ag exhaled by the patient Pt is recirculated so that a ventilation circuit is realized and the anesthetic in the exhaled air Ag does not escape into the environment.

A patient-side fluid guide unit 2 connects the patient-side coupling unit 1 to the base part of a Y-piece 7. The ventilation gas mixture Bg is conveyed through an inspiratory fluid guide unit with segments 3.1, 3.2, and 3.3, as well as through one branch of the Y-piece 7 and the patient-side fluid guide unit 2, to the patient-side coupling unit 1.

The air Ag exhaled by the patient Pt flows through the patient-side coupling unit 1, the other branch of the Y-piece 7, and an expiratory fluid guide unit 4.1, 4.2, 4.3 back to the segment 3.1. A CO2 absorber 18 in segment 4.3 reduces the proportion of carbon dioxide (CO2) in the exhaled air Ag so that exhaled air with a reduced CO2 proportion (Ag—CO2) is fed back into the inspiratory fluid guide unit 3.1, 3.2, 3.3.

A PEEP valve 14 (PEEP=positive end-expiratory pressure) in the segment 4.2 maintains a sufficiently high pressure in the expiratory fluid guide unit 4.1, 4.2, 4.3 and reduces the risk of the patient Pt's lungs collapsing. A pressure relief valve 8 opens if the pressure in the ventilation circuit exceeds an upper threshold. Excess air Ag is conducted through an expiratory discharge fluid guide unit 9 to a sink 10, for example to a waste gas processor 10. The waste gas processor 10 extracts anesthetics from the air Ag so that the extracted anesthetics can be reused.

A volume flow sensor 16 measures the volume flow Vol′3.2 through the segment 3.2. A pressure sensor 17 measures the pressure near the patient-side coupling unit 1, for example in segment 3.3. The measured pressure is approximately the airway pressure (PAW).

A valve arrangement 5 with at least one controllable valve is arranged between the segments 3.1 and 3.2 of the inspiratory fluid guide unit. A fluid conveying unit, in the shown example a pump or a blower 6, generates a uniform pressure in the first segment 3.1. Alternatively, the fluid conveying unit 6 is arranged in segment 4.2 of the expiratory fluid guide unit.

A signal-processing control unit 34 receives a signal from the volume flow sensor 16, a signal from the pressure sensor 17, and a signal from each of the optional, not shown additional sensors, processes the signals received, and controls the valve arrangement 5. By controlling the valve arrangement 5, a sequence of ventilation strokes is effected. In each ventilation stroke, a respective quantity of the ventilation gas mixture Bg is conveyed to the patient-side coupling unit 1. In one embodiment, the control unit 34 performs a closed-loop control, wherein the control objective is that the actual airway pressure PAW and/or the actual volume flow Vol′3.2 to the patient-side coupling unit 1 is equal to a given time course.

A housing 15 of a ventilator 100 surrounds the segments 3.1 and 3.2 of the inspiratory fluid guide unit, segments 4.2 and 4.3 of the expiratory fluid guide unit, the fluid conveying unit 6, the valve arrangement 5, the PEEP valve 14, the pressure relief valve 8, the CO2 absorber 18, the sensor 16, and the control unit 34. The segments 3.3 and 4.1, the Y-piece 7, the patient-side fluid guide unit 2 and the patient-side coupling unit 1, as well as a part of the expiratory discharge fluid guide unit 9 and the sink 10, are arranged outside the housing 15.

An anesthetic dosing unit 20 is inserted into a receptacle (not shown) in the housing 15 and can be removed from this receptacle. The anesthetic dosing unit 20 generates an anesthetic gas mixture Ng and makes it available at a dosing unit outlet 25. The anesthetic gas mixture Ng is fed from the dosing unit outlet 25 through a fluid guide unit 11 to a feed-in point 22 and fed into the first segment 3.1 and thus into the ventilation circuit at the feed-in point 22. Remark: It is possible that several feed-in points 22 are arranged in series. This may help to better distribute the injected anesthetic gas mixture Ng.

The anesthetic dosing unit 20 comprises an anesthetic dosing device (anesthetic dispenser device) 21 and an anesthetic container 23 containing liquid anesthetic. A carrier gas source 13 provides a carrier gas Tg for the anesthetic gas mixture Ng. The provided carrier gas Tg comprises breathing air, pure oxygen, and/or nitrous oxide (N₂O). In the embodiment shown, the carrier gas source 13 is arranged in a wall W. It is also possible that there are several supply connections for the components of the carrier gas Tg in the wall W or in a ceiling supply unit and that a carrier gas mixer generates the carrier gas Tg from at least two of these supplied carrier gas components.

The carrier gas Tg is supplied through a carrier gas fluid guide unit 12 to an inlet of the anesthetic dosing device 21. The anesthetic dosing device 21 conveys anesthetics into a stream of carrier gas Tg, thereby generating the anesthetic gas mixture Ng. In one embodiment, a schematically shown heating element 24 vaporizes or evaporates the liquid anesthetic, and in a chamber not shown, the vaporized or evaporated anesthetic mixes with the carrier gas Tg. In another embodiment, a pump (not shown) injects liquid or gaseous anesthetic into the carrier gas stream Tg.

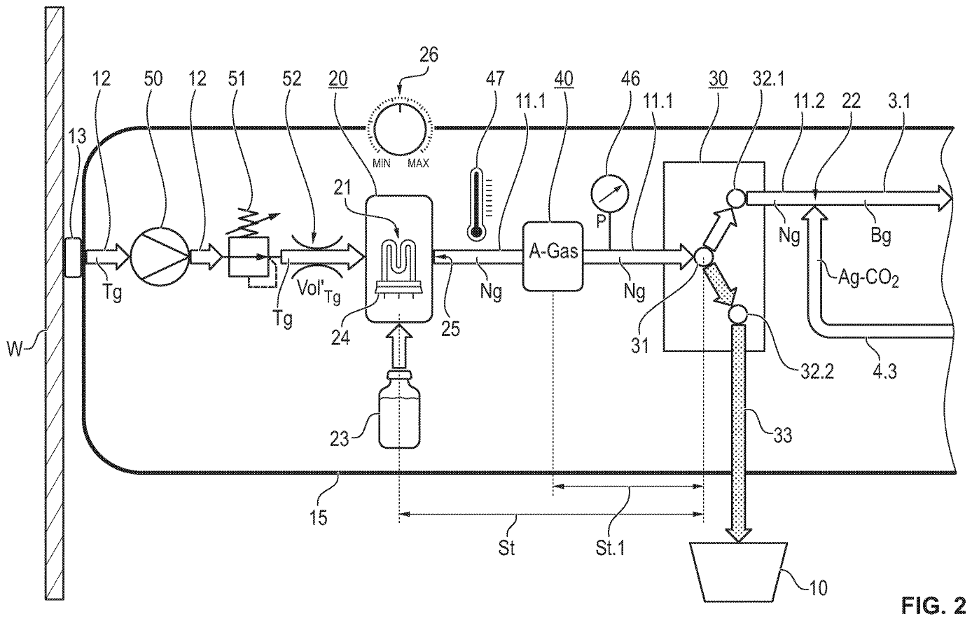

FIG. 2 shows a detail of the ventilation arrangement of FIG. 1. The same reference symbols have the same meanings.

The following components are arranged in the carrier gas fluid guide unit 12 between the carrier gas source 13 and the anesthetic dosing unit 20:
a fluid conveying unit 50,
a controllable proportional valve 51, and
a volume flow sensor 52.

In the embodiment shown, the fluid conveying unit 50 is arranged in the carrier gas fluid guide unit 12 and causes carrier gas Tg to flow from the carrier gas source 13 to the anesthetic dosing unit 20. The control unit 34 controls the proportional valve 51. Depending on this control, the proportional valve 51 controls the volume flow Vol′_{Tg} of the carrier gas Tg through the carrier gas fluid guide unit 12. Thanks to the proportional valve 51, it is not necessary to control the fluid conveying unit 50 in order to change the carrier gas volume flow Vol′_{Tg}. It is also possible that the fluid conveying unit 50 can be controlled, for example by arranging the proportional valve 51 inside the fluid conveying unit 50. The volume flow sensor 52 measures the actual volume flow Vol′_{Tg} of the carrier gas Tg through the carrier gas fluid guide unit 12. It is also possible that the carrier gas source 13 supplies the carrier gas Tg at an overpressure so that no fluid conveying unit 50 is required in the carrier gas fluid guide unit 12.

A pneumatic switch 30 with an inlet 31, a supply outlet 32.1, and a discharge outlet 32.2 is arranged in the supply fluid guide unit 11. The supply fluid guide unit 11 is divided into a first segment 11.1 and a second segment 11.2. The first segment 11.1 leads from the dosing unit outlet 25 to the inlet 31 of the switch 30. The second segment 11.2 leads from the supply outlet 32.1 to the feed-in point 22. A discharge fluid guide unit 33 connects the discharge outlet 32.2 to a sink, for example to the waste gas processor 10 of FIG. 1.

Depending on its position, the switch 30 directs the anesthetic gas mixture Ng from the inlet 31 to the supply outlet 32.1 (the switch 30 is in the supply position) or to the discharge outlet 32.2 (the switch 30 is in the discharge position). The control unit 34 is configured for controlling the pneumatic switch 30 and thereby changing the position of the pneumatic switch 30.

In one embodiment, a temperature sensor 47, an anesthetic sensor 40 (labeled A-Gas), and a pressure sensor 46 are arranged in the first segment 11.1. The temperature sensor 47 measures the temperature of the anesthetic gas mixture Ng flowing through the first segment 11.1. The anesthetic sensor 40 measures the concentration of anesthetic in the anesthetic gas mixture Ng. The pressure sensor 46 measures the pressure in the first segment 11.1. The order (sequence) of these components shown is to be understood as an example only. It is possible that a further anesthetic sensor (not shown) is arranged in the first segment 3.1.

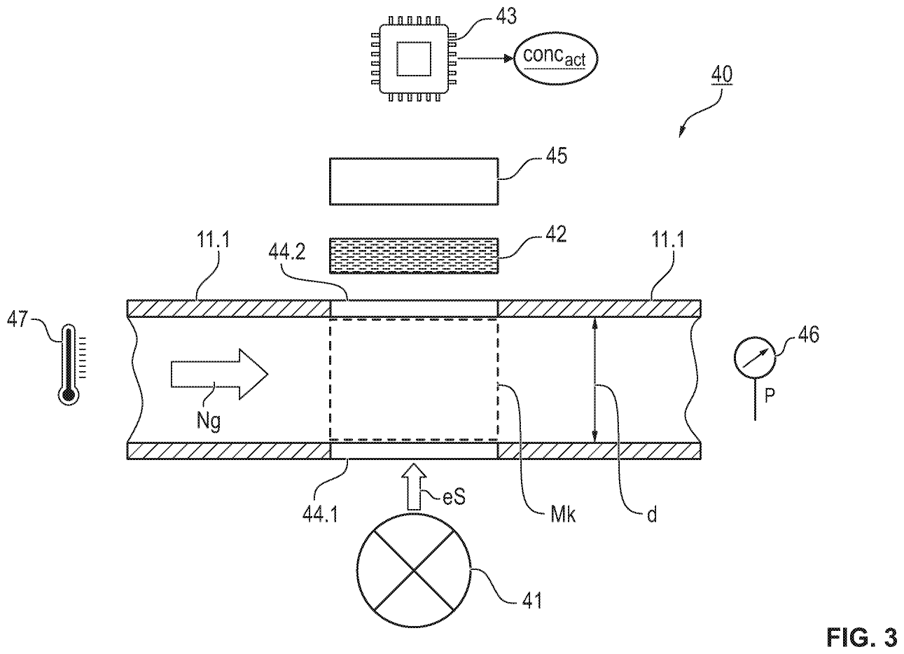

FIG. 3 shows an exemplary embodiment of the anesthetic sensor 40 in the first segment 11.1. According to this embodiment, the anesthetic sensor 40 is a photoelectric sensor. A section of the first segment 11.1 functions as a measuring chamber Mk. The diameter d of this section Mk is, for example, between 3 mm and 4 mm. The anesthetic sensor 40 can also use a process of photoacoustic spectroscopy, non-dispersive infrared measurement, mass spectrometry (mass chromatography), or gas chromatography.

A radiation source 41 emits electromagnetic radiation eS, shown in FIG. 3 as vertically upwards. The emitted electromagnetic radiation eS passes through a first window 44.1 in the wall of segment 11.1, then through the measuring chamber Mk, then through a second window 44.2 and then through a wavelength filter 42, finally striking a photodetector 45. Ideally, the two windows 44.1 and 44.2 are impermeable to fluids and do not react chemically with the anesthetic gas mixture Ng. The photodetector 45 generates a signal that depends on the intensity of the incident electromagnetic radiation eS. Anesthetics in the anesthetic gas mixture Ng attenuate electromagnetic radiation eS in a specific wavelength range as this radiation eS penetrates the anesthetic gas mixture Ng. The attenuation is an indicator of the desired anesthetic concentration. Ideally, the wavelength filter 42 only allows electromagnetic radiation eS in this wavelength range to pass through, thereby reducing the risk of water droplets and particles in the anesthetic gas mixture Ng falsifying the measurement. A signal-processing evaluation unit 43 receives a respective signal from each of the temperature sensor 47, the photodetector 45, and the pressure sensor 46 and derives the actual anesthetic concentration conc_{act} which is to be measured.

In one embodiment, the evaluation unit 43 derives an upper concentration threshold for the concentration of the anesthetic in the anesthetic gas mixture Ng from the measured temperature and the measured pressure in the first segment 11.1. At the measured pressure and at the measured temperature, the concentration of the anesthetic in the anesthetic gas mixture Ng can be at most equal to the derived upper concentration threshold (saturation threshold). The evaluation unit 43 uses the upper concentration threshold in particular for the following two tests:
The possibility that liquid, and thus also anesthetic, condenses on an inner wall of the first segment 11.1 is taken into account. Liquid condenses in particular if the ambient temperature is only slightly above or even below the boiling point of the anesthetic. Condensed anesthetic does not contribute to the anesthetic concentration conc_{act} measured by the anesthetic sensor 40, but can still flow as part of the ventilation gas mixture Bg to the patient-side coupling unit 1. The evaluation unit 43 computationally increases the measured anesthetic concentration conc_{act} if the measurement result of the anesthetic sensor 40 is only slightly (by less than a given tolerance) below the upper concentration threshold.
If the measured anesthetic concentration conc_{act} is more than one tolerance above the upper concentration threshold, at least one sensor 47, 40, 46 has delivered an incorrect measurement result.

In one embodiment, the control unit 34 causes the switch 30 to be switched to the discharge position if it has been detected that at least one sensor 47, 40, 46 delivers an incorrect measurement result. In addition, a corresponding message is output.

In the example shown, the anesthetic sensor 40 is arranged in the first segment 11.1. The anesthetic sensor 40 can also be arranged closer to the patient-side coupling unit 1, for example in the inspiratory fluid guide unit 3.1, 3.2.

The anesthetic dosing unit 20 receives a specification that specifies a desired concentration of the anesthetic originating from the anesthetic container 23 in the generated anesthetic gas mixture Ng, for example as a volume percent (vol. %). This specification is referred to below as the dosing specification. In one embodiment, the maximum possible anesthetic concentration is 2 volume percent. The dosing specification can originate from a user or from a higher-level control system. FIG. 2 shows an example of an input unit in the form of an adjustment wheel 26, which a user can use to specify (set) a desired anesthetic concentration. At the start of the anesthesia of the patient Pt, an initial anesthetic concentration concᵢₙᵢ is specified. It is possible that a different anesthetic concentration is specified at least once during the course of the anesthesia. The desired anesthetic concentration is referred to below as conc_{req}.

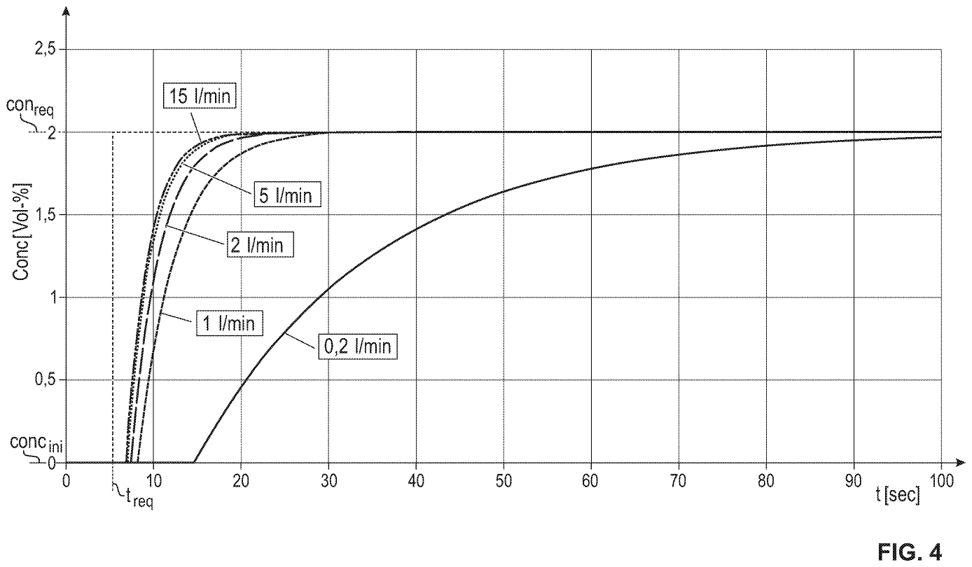

FIG. 4 illustrates the step response of the anesthetic dosing unit 20 to a desired increased anesthetic concentration conc_{req}. The x-axis shows time t in [sec], while the y-axis shows the actual anesthetic concentration Conc_{act} in [vol. %] in the anesthetic gas mixture Ng that the anesthetic dosing unit 20 provides at the dosing unit outlet 25. Before the anesthetic dosing unit 20 captured the dosing specification, the anesthetic dosing unit 20 had not fed any anesthetic into the carrier gas Tg, i.e., concᵢₙᵢ=0. The dosing specification specifies that the anesthetic concentration in the anesthetic gas mixture Ng should now be conc_{req}=2 vol. % and was captured at a time point t_{req}. The diagram shows several time courses of the measured actual anesthetic concentration. The different time courses refer to different values of the volume flow Vol′_{Tg} in [l/min] of the carrier gas Tg to and through the anesthetic dosing unit 20. As can be seen in FIG. 4, the desired anesthetic concentration conc_{req} of 2 vol. % is reached more quickly the higher the volume flow of the carrier gas Tg to the anesthetic dosing unit 20. The anesthetic dosing unit 20 can be described as a PT1 element (Proportional+First-Order Time element—the dosing unit has first-order dynamic behavior).

As shown in FIG. 4, for example, a certain time span elapses between
the process of capturing the dosing specification and
the event that the actual anesthetic concentration conc_{act} downstream of the anesthetic dosing unit 20 matches the specified anesthetic concentration conc_{req} within a given tolerance Tol.

The two embodiments described below result in the anesthetic gas mixture Ng, and therefore also the ventilation gas mixture Bg, only having an anesthetic concentration that deviates from the specification conc_{req} in the dosing specification by more than the tolerance Tol for a relatively short time. A deviation threshold Δ0 is given as an absolute or percentage deviation between two anesthetic concentrations. The steps described below are performed if the desired anesthetic concentration conc_{req} according to the dosing specification deviates by more than the deviation threshold Δ0 from the anesthetic concentration concᵢₙᵢ before the dosing specification was captured.

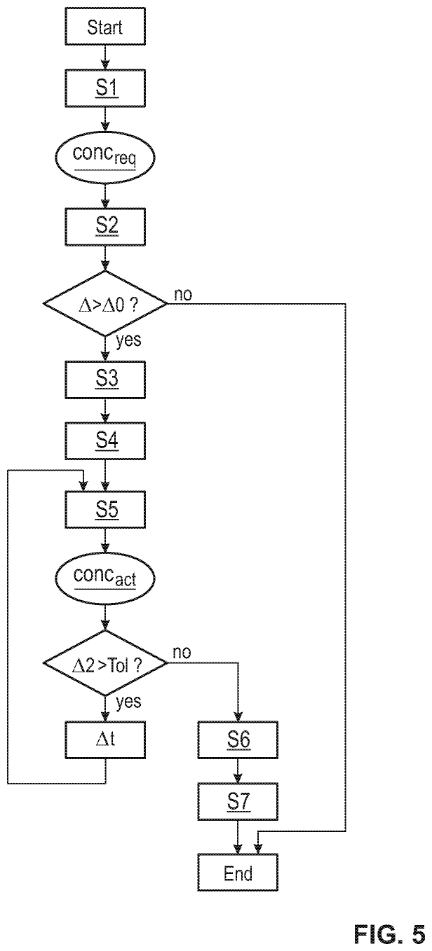

First, the first embodiment is described, which shows how to respond to capturing a dosing specification, with reference to the flowchart in FIG. 5. Before the dosing specification is captured, the pneumatic switch 30 is switched so that it connects the inlet 31 to the supply outlet 32.1. The anesthetic gas mixture Ng has an initial anesthetic concentration concᵢₙᵢ, and the carrier gas Tg is conveyed through the carrier gas fluid guide unit 12 with an initial volume flow vol′ᵢₙᵢ. The elements of the flowchart have the following meanings:

| | |
|---|---|
| S1 | A dosing specification is captured. This dosing specification specifies a |
| | desired anesthetic concentration conc_{req}. |
| S2 | The anesthetic dosing unit 20 responds to the capturing of the dosing |
| | specification with the objective of now providing the anesthetic gas |
| | mixture Ng with the anesthetic concentration conc_{req}. |
| Δ1 > Δ0? | Does the desired anesthetic concentration conc_{req} deviate from the initial |
| | anesthetic concentration concᵢₙᵢ by more than Δ0? In other words, is Δ1 := |
| | \| conc_{req} − concᵢₙᵢ \| > Δ0? |
| S3 | The position of switch 30 is changed by means of a corresponding control |
| | signal from control unit 34. Switch 30 now connects inlet 31 to discharge |
| | outlet 32.2. |
| S4 | The volume flow Vol′_{Tg} of the carrier gas Tg through the carrier gas fluid |
| | guide unit 12 to the anesthetic dosing unit 20 is increased to a value. |
| | Preferably, the proportional valve 51 is controlled accordingly. In one |
| | embodiment, the carrier gas volume flow Vol′_{Tg} is increased to the |
| | maximum possible value vol′ₘₐₓ. |
| S5 | The anesthetic sensor 40 measures the actual anesthetic concentration |
| | conc_{act} in the anesthetic gas mixture Ng. |
| Δ2 > Tol? | Does the measured actual anesthetic concentration conc_{act} deviate from |
| | the specified anesthetic concentration conc_{req} by more than the tolerance |
| | Tol? In other words, is Δ2 = \| conc_{act} − conc_{req} \| > Tol? |
| Δt | A sampling time span Δt elapses. |
| S6 | The position of switch 30 is changed again by means of a corresponding |
| | control signal. Switch 30 now connects inlet 31 to supply outlet 32.1. |
| S7 | The flow of carrier gas Tg through the carrier gas fluid guide unit 12 is |
| | reduced back to the old value vol′ᵢₙᵢ. |

Step S3 (the pneumatic switch 30 is switched) causes the anesthetic gas mixture Ng to be conveyed through the discharge fluid guide unit 33 to the sink 10. Step S4 (carrier gas volume flow is increased) causes the supply fluid guide unit 11 to be quickly flushed out. The increased volume flow vol′ₘₐₓ of the carrier gas Tg also causes the anesthetic dosing unit 20 to supply the anesthetic gas mixture Ng more quickly with the desired anesthetic concentration conc_{req} compared to a constant carrier gas volume flow vol′ᵢₙᵢ, see FIG. 4.

The first embodiment described with reference to FIG. 5 requires the anesthetic sensor 40. Step S4, in which the volume flow Vol′_{Tg} of the carrier gas Tg is increased, and step S7, in which the carrier gas volume flow Vol′_{Tg} is reduced again, can be performed by a closed-loop control, i.e., depending on a signal from the volume flow sensor 52. It is also possible to perform steps S4 and S7 by an open-loop control and not necessarily to use the volume flow sensor 52.

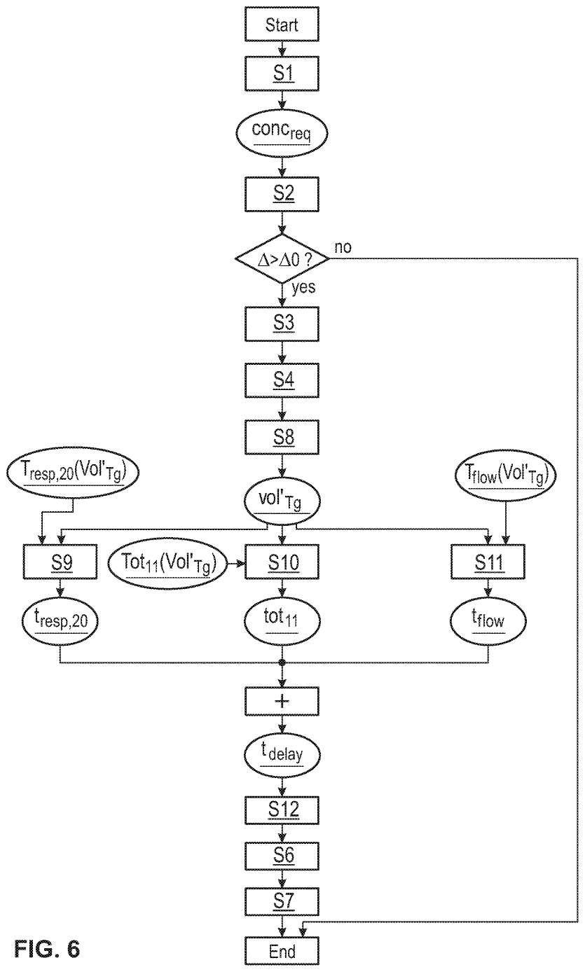

A second embodiment is described below with reference to FIG. 6. The same reference symbols have the same meaning as in FIG. 5. The second embodiment does not require an anesthetic sensor 40.

According to the second embodiment, a time span is determined which elapses between the following two events:
The dosing specification is captured (step S1).
The anesthetic gas mixture Ng has the specified anesthetic concentration conc_{req} at a reference point, up to the given tolerance Tol. The reference point is arranged in the first segment 11.1 and, according to the second embodiment, is the inlet 31 of the switch 30, but can also be another point between the dosing unit outlet 25 and the inlet 31 in the first segment 11.1.

Depending on the determined time span, after capturing the dosing specification, a prediction is made as to when the anesthetic gas mixture Ng at the reference point 31 will have the specified anesthetic concentration conc_{req}.

In a preferred embodiment, the following is determined in advance:

The response time T_{resp,20}=T_{resp,20}(Vol′_{Tg}) describes the response behavior of the anesthetic dosing unit 20 in reaction to capturing the dosing specification. The response time T_{resp,20} depends on the volume flow value vol′_{Tg} to the anesthetic dosing unit 20 and, optionally, also on the deviation Δ1 between the specified anesthetic concentration conc_{req} and the initial anesthetic concentration concᵢₙᵢ, i.e., T_{resp,20}=T_{resp,20}(Vol′_{Tg}, Δ1). More precisely, the response time T_{resp,20} elapses between the following two events:
The dosing specification is captured.
The anesthetic gas mixture Ng has an actual anesthetic concentration conc_{act} at the dosing unit outlet 25, which deviates by a maximum of x %, i.e., by no more than the tolerance Tol, from the specified anesthetic concentration conc_{req}, wherein x is given.

In one embodiment, the anesthetic dosing unit 20 can be described as a PT1 element, see FIG. 4. The response time T_{resp,20} is, for example, the time span that elapses from an initial anesthetic concentration concᵢₙᵢ of zero until a value of ×1% of the specified anesthetic concentration conc_{req} is reached at the dosing unit outlet 25, where, for example, ×1=90 [%].

A functional relationship Tot₁₁=Tot₁₁(Vol′_{Tg}) describes a pneumatic dead time tot₁₁ at least as a function of the volume flow Vol′_{Tg} with which the carrier gas Tg is conveyed through the carrier gas fluid guide unit 12 to the anesthetic dosing unit 20. The pneumatic dead time tot₁₁ depends on the volume flow value vol′_{Tg} and on the volume and geometry of the first segment 11.1 along a second section St from the dosing unit outlet 25 to the reference point 31, in particular to the inlet 31 of the switch 30. The volume and geometry of the first segment 11.1 generally remain constant while the ventilation arrangement is in use. Optionally, other parameters may occur in the functional context of dead time Tot₁₁ on which the pneumatic dead time tot₁₁ depends, for example the temperature and/or pressure in the first segment 11.1. The pneumatic dead time tot₁₁ is the time span in which the fluid guide unit (the pneumatic path) is flushed from the dosing unit outlet 25 of the anesthetic dosing unit 20 to the reference point 31. In other words: The pneumatic dead time tot₁₁ is the time span that elapses between the following two events:
The dosing specification is captured.
The gas mixture that was located in the fluid guide unit between the dosing unit outlet 25 and the reference point 31 in the first segment 11.1 when the dosing specification was captured is now located downstream of this reference point 31, preferably downstream of the first segment 11.1.

The response time T_{resp,20} describes after what time span the anesthetic concentration at the dosing unit outlet 25 in the anesthetic gas mixture Ng matches the specified anesthetic concentration conc_{req} sufficiently accurately. However, the anesthetic gas mixture Ng with this anesthetic concentration conc_{req} does not immediately reach the reference point 31. Instead, the anesthetic gas mixture Ng flows from the dosing unit outlet 25 through segment 11.1 to the inlet 31 and inevitably reaches the reference point 31 only after a flow time T_{flow}=T_{flow}(Vol′_{Tg}). This flow time T_{flow} is also determined empirically in advance. The flow time T_{flow} is preferably the time span between the following two events:
After the dosing specification has been captured at the dosing unit outlet 25, the anesthetic gas mixture Ng has a specific anesthetic concentration concₓ.
The anesthetic gas mixture Ng has this anesthetic concentration concₓ at the reference point 31, in particular at the inlet 31.

According to the second embodiment, the step in which the dosing specification is captured triggers the following step: A prediction is made as to when the anesthetic gas mixture Ng at reference point 31 will have the specified anesthetic concentration conc_{req} with sufficient accuracy. “With sufficient accuracy” means that the deviation between the actual and specified anesthetic concentration is not greater than a given tolerance Tol. The prediction depends at least on the volume flow value vol′_{Tg} of the carrier gas Tg through the carrier gas fluid guide unit 12. The volume flow through the first segment 11.1 corresponds sufficiently accurately to the volume flow Vol′_{Tg} specified by the carrier gas source 13. In one embodiment, the volume flow sensor 52 measures the volume flow Vol′_{Tg}. In another embodiment, a specification for the carrier gas source 13 is captured, which specifies the volume flow Vol′_{Tg}. The following description uses the term “the volume flow Vol′_{Tg} is determined,” which refers to both the implementation in which the volume flow sensor 52 measures the volume flow Vol′_{Tg} and the implementation in which a specification for the volume flow Vol′_{Tg} is captured.

Corresponding reference symbols in FIG. 5 and FIG. 6 have the same meanings. The remaining reference symbols in FIG. 6 have the following meanings:

| | |
|---|---|
| S8 | The carrier gas volume flow Vol′_{Tg} is determined. |
| S9 | The calculation rule describing the response behavior of the anesthetic |
| | dosing unit 20 is applied to the determined volume flow Vol′_{Tg}. |
| T_{resp, 20} | Dependence of the response time of the anesthetic dosing unit 20 on |
| | the volume flow Vol′_{Tg} |
| t_{resp, 20} | Response time of the anesthetic dosing unit 20 that occurs at the |
| | volume flow value vol′_{Tg} |
| S10 | The functional relationship Tot₁₁(Vol′_{Tg}), which describes the |
| | pneumatic dead time of the first segment 11.1, is applied to the |
| | determined volume flow value vol′_{Tg}. |
| tot₁₁ | Pneumatic dead time of the first segment 11.1 that occurs at the |
| | volume flow value vol′_{Tg}. |
| S11 | The functional relationship T_{flow}(Vol′_{Tg}), which describes the flow time |
| | of the anesthetic gas mixture Ng through the first segment 11.1, is |
| | applied to the determined volume flow value vol′_{Tg}. |
| t_{flow} | Flow time through the first segment 11.1 that occurs at the volume |
| | flow value vol′_{Tg}. |
| t_{delay} | Predicted delay time span, i.e., the time span between capturing the |
| | dosing specification and the event at which the anesthetic gas mixture |
| | Ng at reference point 31 approximately reaches the specified anesthetic |
| | concentration conc_{req} |
| S12 | The predicted delay time span t_{delay} elapses. |

In the implementation shown in FIG. 6, three delay parameters, that are dependent on the carrier gas volume flow Vol′_{Tg}, are determined, namely the response time t_{resp,20} of the anesthetic dosing unit 20, the pneumatic dead time tot₁₁, and the flow time t_{flow}. In addition, in the embodiment shown, the inlet 31 of the switch 30 is used as a reference point, so that the pneumatic dead time tot₁₁ and the flow time t_{flow} refer to the entire first segment 11.1.

In a different embodiment, only one delay parameter is determined. The inventors have found in internal tests that the pneumatic dead time tot₁₁ has a greater influence on the delay time span t_{delay} than the other two delay parameters t_{resp,20} and t_{flow}. Therefore, in a different implementation, only the functional relationship Tot₁₁(Vol′_{Tg}) and, in step S10, the pneumatic dead time tot₁₁ are determined in advance. It is also possible that the pneumatic dead time tot₁₁ and, in addition, one of the other two delay parameters t_{resp,20} or t_{flow} are determined.

The implementation shown in FIG. 6 does not require an anesthetic sensor 40. In a third embodiment, an anesthetic sensor 40 is used as described for the first embodiment with reference to FIG. 5, and in addition, a different implementation of the second embodiment described with reference to FIG. 6 is used.

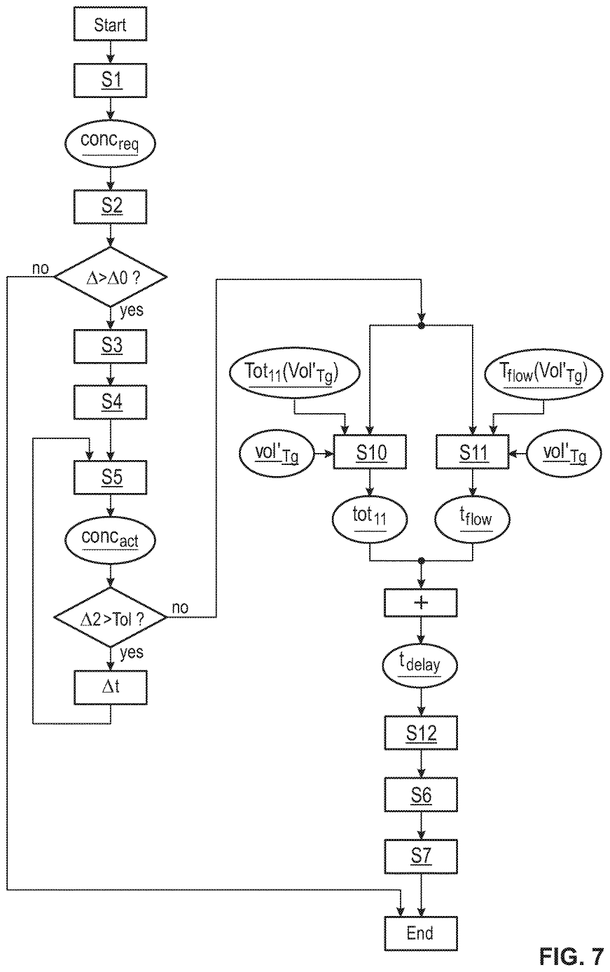

The third embodiment is described below with reference to FIG. 7. The reference point is arranged downstream of the anesthetic sensor 40, in particular downstream of the measuring chamber Mk according to the embodiment of FIG. 3. Preferably, the inlet 31 functions as the reference point. Because the actual anesthetic concentration conc_{act} is measured in the first segment 11.1, it is not necessary to determine the response time t_{resp,20}. Therefore, step S9 of FIG. 6 is also omitted. The functional relationships Tot₁₁(Vol′_{Tg}) and T_{flow} (Vol′_{Tg}), the pneumatic dead time tot₁₁ and the flow time t_{flow} refer, according to the third embodiment, to a first section St.1 in the first segment 11.1 from the anesthetic sensor 40, in particular from the measuring chamber Mk, to the reference point, in particular to the inlet 31 of the switch 30.

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | Patient-side coupling unit, attached in and/or on the body of the |
| | patient Pt |
| 2 | Patient-side fluid guide unit, connects the patient-side coupling unit |
| | 1 to the Y-piece 7 |
| 3.1, 3.2 | Segments of the inspiratory fluid guide unit in housing 15 |
| 3.3 | Segment of the inspiratory fluid guide unit between the housing 15 |
| | and the Y-piece 7 |
| 4.1 | Segment of the expiratory fluid guide unit between housing 15 and |
| | Y-piece 7 |
| 4.2, 4.3 | Segments of the expiratory fluid guide unit in housing 15 |
| 5 | Controllable valve arrangement between segments 3.1 and 3.2, |
| | causes the ventilation strokes |
| 6 | Fluid conveying unit in the form of a pump or blower in the first |
| | segment 3.1 |
| 7 | Y-piece connecting the patient-side fluid guide unit 2 to segments |
| | 3.3 and 4.1 |
| 8 | Pressure relief valve in the expiratory fluid guide unit, arranged |
| | between segments 4.1 and 4.2 |
| 9 | Expiratory discharge fluid guide unit, which guides excess exhaled |
| | air Ag from the pressure relief valve 8 to the sink 10 |
| 10 | Sink (waste gas processor) for excess exhaled air Ag and excess and |
| | discharged anesthetic gas mixture Ng |
| 11 | Supply fluid guide unit, which guides the anesthetic gas mixture Ng |
| | from the dosing unit outlet 25 of the anesthetic dosing unit 20 to the |
| | feed-in point 22 |
| 11.1 | First segment of the supply fluid guide unit 11, directs the anesthetic |
| | gas mixture Ng from the dosing unit outlet 25 of the anesthetic |
| | dosing unit 20 to the inlet 31 of the pneumatic switch 30. |
| 11.2 | Second segment of the supply fluid guide unit 11, which directs the |
| | anesthetic gas mixture Ng from the supply outlet 32.1 of the switch |
| | 30 to the feed-in point 22. |
| 12 | Carrier gas fluid guide unit, directs the carrier gas Tg from the |
| | carrier gas source 13 to the anesthetic dosing unit 20 |
| 13 | Source for a carrier gas Tg, arranged in the wall W |
| 14 | PEEP valve in segment 4.2 |
| 15 | Housing of the ventilator 100 |
| 16 | Volume flow sensor, measures the volume flow Vol′_{3.2} of the |
| | ventilation gas mixture Bg through segment 3.2 |
| 17 | Pressure sensor, measures the airway pressure P_{AW} |
| 18 | CO2 absorber in segment 4.3 |
| 20 | Anesthetic dosing unit, comprising the anesthetic dosing device 21 |
| | and the anesthetic container 23, generates the anesthetic gas mixture |
| | Ng and makes it available at the dosing unit outlet 25. |
| 21 | Anesthetic dosing device of the anesthetic dosing unit 20, feeds |
| | anesthetic into a stream of carrier gas Tg |
| 22 | Feed-in point at which the anesthetic gas mixture Ng is fed into the |
| | ventilation circuit. |
| 23 | Anesthetic container of the anesthetic dosing unit 20, contains liquid |
| | anesthetic. |
| 24 | Heating element of the anesthetic dosing unit 20, evaporates liquid |
| | anesthetic |
| 25 | Dosing unit outlet of the anesthetic dosing unit 20, also the start of |
| | the first segment 11.1 |
| 26 | Input unit with which a user can specify a desired anesthetic |
| | concentration in the anesthetic gas mixture Ng |
| 30 | Pneumatic switch, comprises the switch inlet 31 and the outlets 32.1, |
| | 32.2, is controlled by the control unit 34. |
| 31 | Switch inlet of switch 30, functions as a reference point in one |
| | embodiment |
| 32.1 | Supply outlet of switch 30, start of the second segment 11.2 |
| 32.2 | Discharge outlet of switch 30, start of discharge fluid guide unit 33 |
| 33 | Discharge fluid guide unit, guides the anesthetic gas mixture Ng |
| | from the discharge outlet 32.2 of the switch 30 to the sink 10 |
| 34 | Signal processing control unit, controls the valve arrangement 5 and |
| | the pneumatic switch 30 |
| 40 | Anesthetic sensor, measures the concentration of the anesthetic in |
| | the anesthetic gas mixture Ng, comprises the measuring chamber |
| | Mk |
| 41 | Radiation source of the anesthetic sensor 40 |
| 42 | Wavelength filter of the anesthetic sensor 40 |
| 43 | Signal processing evaluation unit of the anesthetic sensor 40 |
| 44.1, 44.2 | Window of the measuring chamber Mk |
| 45 | Photodetector of the anesthetic sensor 40 |
| 46 | Pressure sensor, measures the pressure of the anesthetic gas mixture |
| | Ng in the first segment 11.1 |
| 47 | Temperature sensor, measures the temperature of the anesthetic gas |
| | mixture Ng in the first segment 11.1 |
| 50 | Fluid conveying unit, conveys carrier gas Tg from the carrier gas |
| | source 13 through the carrier gas fluid guide unit 12 |
| 51 | Proportional valve, controls the volume flow Vol′_{Tg} of carrier gas Tg |
| | through the carrier gas fluid guide unit 12 |
| 52 | Volume flow sensor, measures the volume flow Vol′_{Tg} of carrier gas |
| | Tg through the carrier gas fluid guide unit 12 |
| 100 | Ventilator, comprises the housing 15, the segments 3.1, 3.2, 4.2, 4.3, |
| | the fluid conveying unit 6, the valve arrangement 5, the PEEP valve |
| | 14, the pressure relief valve 8, and the CO2 absorber 18, is |
| | connected to the anesthetic dosing unit 20 |
| Ag | Air exhaled by the patient Pt is conveyed by the patient-side |
| | coupling unit 1 through the expiratory fluid guide unit 4 to the feed- |
| | in point 22. |
| Ag—CO2 | Exhaled air Ag downstream of the CO2 absorber 18 |
| Bg | Ventilation gas mixture, is conveyed through the inspiratory fluid |
| | guide unit 3 to the patient-side coupling unit 1, comprises the |
| | anesthetic gas mixture Ng |
| conc_{act} | Actual anesthetic concentration in the anesthetic gas mixture Ng, |
| | measured by the anesthetic sensor 40 |
| concᵢₙᵢ | Anesthetic concentration before capturing the dosing specification |
| conc_{req} | Desired anesthetic concentration specified in the dosing |
| | specification |
| d | Diameter of the measuring chamber Mk |
| Δ0 | Deviation threshold as the maximum permissible absolute or |
| | percentage deviation between two anesthetic concentrations |
| Δ1 | Deviation between the desired anesthetic concentration conc_{req} and |
| | the initial anesthetic concentration concᵢₙᵢ |
| Δ2 | Deviation between the actual anesthetic concentration conc_{act} and |
| | the desired anesthetic concentration conc_{req} |
| eS | Electromagnetic radiation emitted by radiation source 41 |
| Mk | Measuring chamber of the anesthetic sensor 40, is a section of the |
| | first segment 11.1, has a diameter d |
| Ng | Anesthetic gas mixture, generated by the anesthetic dosing unit 20 |
| | and fed through the supply fluid guide unit 11 to the feed-in point 22 |
| P | Pressure |
| P_{AW} | Airway pressure, measured by the pressure sensor 17 |
| PEEP | Positive end-expiratory pressure |
| Pt | Artificially ventilated (ventilated) patient, wears the patient-side |
| | coupling unit 1 |
| St | Second section in the first segment 11.1 from the dosing unit outlet |
| | 25 to the reference point 31 |
| St. 1 | First section in the first segment 11.1 from the measuring chamber |
| | Mk to reference point 31 |
| t_{delay} | Predicted delay time span |
| T_{flow}(Vol′_{Tg}) | Functional relationship, describes the flow time of the anesthetic gas |
| | mixture Ng through the first segment 11.1 as a function of the |
| | volume flow Vol′_{Tg} |
| t_{flow} | Derived flow time of the anesthetic gas mixture Ng through segment |
| | 11.1 to reference point 31 at volume flow value vol′_{Tg} |
| Tg | Carrier gas, provided by the carrier gas source 13 |
| Tol | Tolerance: permissible deviation between the concentrations conc_{act} |
| | and conc_{req} |
| Tot₁₁(Vol′_{Tg}) | Functional relationship describing the pneumatic dead time tot₁₁ of |
| | the segment 11.1 as a function of the volume flow Vol′_{Tg} |
| tot₁₁ | Pneumatic dead time of segment 11.1 at the volume flow value |
| | vol′_{Tg} |
| T_{resp, 20}(Vol′_{Tg}) | Functional relationship describing the response time of the |
| | anesthetic dosing unit 20 as a function of the volume flow Vol′_{Tg} |
| t_{req} | Time point at which the dosing specification is captured |
| t_{resp, 20} | Response time of the anesthetic dosing unit 20 at the volume flow |
| | value vol′_{Tg} |
| Δt | Sampling time span |
| W | Wall with carrier gas source 13 |
| Vol′_{3.2} | Volume flow through segment 3.2, measured by volume flow sensor |
| | 16 |
| vol′ᵢₙᵢ | Carrier gas volume flow before capturing the dosing specification |
| vol′ₘₐₓ | Maximum achievable carrier gas volume flow |
| Vol′_{Tg} | Volume flow of carrier gas Tg through the carrier gas fluid guide |
| | unit 12, approximately equal to the volume flow of the anesthetic |
| | gas mixture Ng through the first segment 11.1 |
| vol′_{Tg} | Value of the volume flow Vol′_{Tg} |

What is claimed is:

1. A ventilation arrangement for artificially ventilating a patient, the ventilation arrangement comprising:
a patient-side coupling unit, which is configured to be arranged on and/or in the body of the patient;
a carrier gas fluid guide unit;
a supply fluid guide unit;
an inspiratory fluid guide unit;
a discharge fluid guide unit;
an anesthetic dosing unit with a dosing unit outlet;
a pneumatic switch with a switch inlet, a supply outlet, and a discharge outlet, the pneumatic switch being configured to be switched into a supply position in which the switch inlet is connected to the supply outlet and into a discharge position in which the switch inlet is connected to the discharge outlet; and
a signal-processing control unit,
wherein the ventilation arrangement is configured to capture a dosing specification which specifies a desired anesthetic concentration,
wherein the anesthetic dosing unit is configured to generate an anesthetic gas mixture comprising a carrier gas and an anesthetic with an objective that an actual concentration of the anesthetic in the anesthetic gas mixture coincides with the desired anesthetic concentration of the captured dosing specification, and to provide the anesthetic gas mixture at the dosing unit outlet,
wherein the carrier gas fluid guide unit connects a carrier gas source to the anesthetic dosing unit,
wherein a first segment of the supply fluid guide unit connects the dosing unit outlet to the switch inlet of the pneumatic switch and a second segment of the supply fluid guide unit connects the supply outlet of the pneumatic switch to a feed-in point,
wherein the discharge fluid guide unit connects the discharge outlet to a sink,
wherein the inspiratory fluid guide unit connects the feed-in point to the patient-side coupling unit,
wherein the ventilation arrangement is configured to convey the anesthetic gas mixture through the first segment of the supply fluid guide unit to the switch inlet,
wherein the ventilation arrangement is configured, with the pneumatic switch in the supply position, to convey the anesthetic gas mixture through the pneumatic switch to the second segment of the supply fluid guide unit and to the feed-in point, to supply the anesthetic gas mixture into the inspiratory fluid guide unit at the feed-in point, and to convey a ventilation gas mixture comprising the anesthetic gas mixture from the feed-in point through the inspiratory fluid guide unit to the patient-side coupling unit, and with the pneumatic switch in the discharge position, to convey the anesthetic gas mixture through the pneumatic switch to the discharge fluid guide unit and to the sink,
wherein the control unit is configured, in response to capturing the dosing specification, to cause the pneumatic switch to be switched to the discharge position, and to check whether a target situation exists, which target situation exists upon an actual anesthetic concentration at a reference point in the first segment deviating by no more than a given tolerance from the desired anesthetic concentration of the dosing specification, and
wherein the control unit is configured, in response to the detection that the target situation exists, to cause the pneumatic switch to be switched to the supply position.

2. The ventilation arrangement according to claim 1, wherein the reference point is at or adjacent to the switch inlet.

3. The ventilation arrangement according to claim 1, further comprising an anesthetic sensor, which is configured to measure a concentration of the anesthetic in the anesthetic gas mixture at a measuring position as a measured actual anesthetic concentration,
wherein the measuring position is arranged in the first segment, and
wherein the control unit is configured to check, depending on the measured actual anesthetic concentration, if the target situation exists.

4. The ventilation arrangement according to claim 3, wherein the control unit is configured to determine that the target situation exists if the measured actual anesthetic concentration deviates from the specified anesthetic concentration by no more than the given tolerance.

5. The ventilation arrangement according to claim 3,
wherein the control unit is configured to determine a volume flow of the carrier gas through the carrier gas fluid guide unit,
wherein the reference point is arranged downstream of the measuring position,
wherein the control unit is further configured to determine one or more delay parameters of a group of delay parameters comprising:
a pneumatic dead time at the determined volume flow in a first section leading in the first segment from the measuring position of the anesthetic sensor to the reference point, and
a flow time of the anesthetic gas mixture at the determined volume flow through the first section in the first segment, and
the control unit is further configured
to detect an event that the measured actual anesthetic concentration at the measuring position deviates from the specified desired anesthetic concentration by no more than the given tolerance,
to determine a delay time span depending on the determined one or more delay parameters, and
to determine that the target situation exists upon the determined delay time span elapsing since the detection that the actual anesthetic concentration at the measuring position deviates from the specified desired anesthetic concentration by no more than the given tolerance.

6. The ventilation arrangement according to claim 1,
wherein the control unit is configured to measure or otherwise determine the volume flow of the carrier gas through the carrier gas fluid guide unit, and
wherein the control unit is further configured to determine one or more delay parameters of a group of delay parameters comprising:
a response time of the anesthetic dosing unit at the determined volume flow,
a pneumatic dead time at the determined volume flow in a second section leading in the first segment from the dosing unit outlet to the reference point, and
a flow time of the anesthetic gas mixture at the determined volume flow through the second section in the first segment, and
wherein the control unit is further configured
to determine a delay time span depending on the determined one or more delay parameters, and
to determine that the target situation exists if the determined delay time span has elapsed since capturing the dosing specification.

7. The ventilation arrangement according to claim 1, wherein the control unit is configured to, after capturing the dosing specification, increase a volume flow of the carrier gas through the carrier gas fluid guide unit and/or increase a volume flow of the anesthetic gas mixture through the first segment.

8. The ventilation arrangement according to claim 7, wherein the control unit is configured to, after capturing the dosing specification, increase a volume flow of the carrier gas through the carrier gas fluid guide unit to a given maximum achievable value and/or increase a volume flow of the anesthetic gas mixture through the first segment to a given maximum achievable value.

9. The ventilation arrangement according to claim 7, wherein the control unit is configured to, in response to the determination that the target situation exists, reduce the volume flow of the carrier gas and/or reduce the volume flow of the anesthetic gas mixture.

10. A ventilation process for artificially ventilating a patient, the process is performed using a ventilation arrangement comprising a patient-side coupling unit, an anesthetic dosing unit with a dosing unit outlet, a carrier gas fluid guide unit connecting a carrier gas source to the anesthetic dosing unit, a supply fluid guide unit, an inspiratory fluid guide unit, a discharge fluid guide unit, a pneumatic switch with a switch inlet, a supply outlet, and a discharge outlet, wherein the pneumatic switch is configured to be switched into a supply position, in which the switch inlet is connected to the supply outlet, and into a discharge position, in which the switch inlet is connected to the discharge outlet,
wherein the ventilation process is performed while the patient-side coupling unit is arranged on and/or in the body of the patient, the ventilation process comprising the steps of:
with the ventilation arrangement, capturing a dosing specification which specifies a desired anesthetic concentration;
with the anesthetic dosing unit, generating an anesthetic gas mixture comprising a carrier gas and an anesthetic, the generation is performed with an objective that an actual concentration of the anesthetic in the anesthetic gas mixture coincides with the desired anesthetic concentration of the captured dosing specification, and providing the anesthetic gas mixture at the dosing unit outlet,
conveying the anesthetic gas mixture from the dosing unit outlet through a first segment of the supply fluid guide unit to the switch inlet,
with the pneumatic switch in the supply position, conveying the anesthetic gas mixture through the pneumatic switch and through a second segment of the supply fluid guide unit to a feed-in point, supplying the anesthetic gas mixture into the inspiratory fluid guide unit at the feed-in point, and conveying a ventilation gas mixture comprising the anesthetic gas mixture from the feed-in point to the patient-side coupling unit; and
with the pneumatic switch in the discharge position, conveying the anesthetic gas mixture through the pneumatic switch and the discharge fluid guide unit to a sink,
wherein the step of capturing the dosing specification triggers the further steps of:
switching the pneumatic switch to the discharge position; and
with the control unit, determining that a target situation exists, which target situation exists upon an actual concentration of the anesthetic in the anesthetic gas mixture at a reference point in the first segment deviating by no more than a given tolerance from the desired anesthetic concentration specified in the dosing specification, and
wherein the determination that the target situation exists triggers the step of switching the pneumatic switch to the supply position.

11. The ventilation process according to claim 10, wherein the reference point is at or adjacent to the switch inlet.

12. The ventilation process according to claim 10,
wherein the ventilation arrangement comprises an anesthetic sensor and the ventilation process further comprises the step of measuring, with the anesthetic sensor, an actual concentration of the anesthetic in the anesthetic gas mixture at a measuring position, and
wherein the measuring position is arranged in the first segment and the step of checking if the target situation exists is performed depending on the measured actual anesthetic concentration.

13. The ventilation process according to claim 12, wherein it is determined that the target situation exists if the actual anesthetic concentration at the measuring position deviates from the specified desired anesthetic concentration by no more than the given tolerance.

14. The ventilation process according to claim 10, wherein the ventilation process further comprises the steps of:
determining a volume flow of the carrier gas through the carrier gas fluid guide unit;
determining one or more delay parameters of the group of delay parameters comprising:
a response time of the anesthetic dosing unit at the determined volume flow;
a pneumatic dead time in a section in the first segment from the dosing unit outlet to the reference point at the determined volume flow; and
a flow time of the anesthetic gas mixture through the section, in the first segment, at the determined volume flow;
determining a delay time span depending on the determined one or more delay parameters; and
determining that the target situation exists upon the determined delay time span having elapsed since capturing the dosing specification.

15. The ventilation process according to claim 10, wherein the step of capturing the dosing specification additionally triggers the further step of increasing a volume flow of the carrier gas through the carrier gas fluid guide unit and/or increasing a volume flow of the anesthetic gas mixture through the first segment.

16. The ventilation process according to claim 15, wherein the further step of increasing a volume flow of the carrier gas through the carrier gas fluid guide unit increases the volume flow of the carrier gas through the carrier gas fluid guide unit to a given maximum achievable value and/or the step of increasing a volume flow of the anesthetic gas mixture through the first segment increases the volume flow of the anesthetic gas mixture through the first segment to a given maximum achievable value.

17. The ventilation process according to claim 15, wherein the determination that the target situation exists additionally triggers the further step of reducing the volume flow of the carrier gas and/or reducing the volume flow of the anesthetic gas mixture.

18. A ventilation system comprising:
an anesthetic dosing unit with a dosing unit outlet, the anesthetic dosing unit being configured to generate an anesthetic gas mixture comprising a carrier gas and an anesthetic and to provide the anesthetic gas mixture at the dosing unit outlet;
a carrier gas fluid guide unit connecting a carrier gas source to the anesthetic dosing unit;
a pneumatic switch comprising a switch inlet, a supply outlet, and a discharge outlet, the pneumatic switch being configured to be switched into a supply position in which the switch inlet is connected to the supply outlet and into a discharge position in which the switch inlet is connected to the discharge outlet;
a supply fluid guide unit, which comprises a first segment connecting the dosing unit outlet to the switch inlet and a second segment connecting the supply outlet to a feed-in point;
an inspiratory fluid guide unit, which connects the feed-in point to a patient-side coupling unit;
a discharge fluid guide unit, which connects the discharge outlet to a sink; and
a signal-processing control unit, which is configured:
to capture a dosing specification which specifies a desired anesthetic concentration;
to switch the pneumatic switch to the discharge position in response to capturing the dosing specification;
to check if a target situation exists, which target situation exists if an anesthetic concentration in the first segment deviates by no more than a given tolerance from the desired anesthetic concentration; and
to switch the pneumatic switch to the supply position in response to determining that the target situation exists,
wherein the ventilation system is configured, with the pneumatic switch in the supply position, to guide the anesthetic gas mixture through the first segment to the switch inlet, through the pneumatic switch to the second segment and to supply the anesthetic gas mixture to the feed-in point and into the inspiratory fluid guide unit, and
wherein the ventilation system is configured, with the pneumatic switch in the discharge position, to guide the anesthetic gas mixture through the pneumatic switch to the discharge fluid guide unit and to the sink.

19. The ventilation system according to claim 18, further comprising an anesthetic sensor, which is configured to measure a concentration of the anesthetic in the anesthetic gas mixture at a measuring position in the first segment as a measured anesthetic concentration,
wherein the control unit is further configured to check if the measured anesthetic concentration deviates from the desired anesthetic concentration by no more than the given tolerance,
wherein the control unit is further configured to determine a volume flow of the carrier gas through the carrier gas fluid guide unit,
wherein the control unit is further configured to determine one or more delay parameters of a group of delay parameters comprising:
a pneumatic dead time at the determined volume flow in a first section leading in the first segment from the measuring position to the pneumatic switch, and
a flow time of the anesthetic gas mixture at the determined volume flow through the first section in the first segment, and
wherein the control unit is further configured
to determine a delay time span depending on the determined one or more delay parameters, and
to determine that the target situation exists upon the determined delay time span elapsing since the control unit determined that the measured anesthetic concentration at the measuring position deviates from the specified desired anesthetic concentration by no more than the given tolerance.

20. The ventilation system according to claim 18,
wherein the control unit is further configured to determine a volume flow of the carrier gas through the carrier gas fluid guide unit as a determined volume flow, and
wherein the control unit is further configured to determine one or more delay parameters of a group of delay parameters comprising:
a response time of the anesthetic dosing unit at the determined volume flow,
a pneumatic dead time at the determined volume flow in a second section leading in the first segment from the dosing unit outlet to the pneumatic switch, and
a flow time of the anesthetic gas mixture at the determined volume flow through the second section in the first segment, and
wherein the control unit is further configured
to determine a delay time span depending on the determined one or more delay parameters, and
to determine that the target situation exists if the determined delay time span has elapsed since capturing the dosing specification.
